(19) United States
(12) Patent Application Publication
(10) Pub. No.: US 2010/0059068 A1
(43) Pub. Date: Mar. 11, 2010

(54) Fixation Wrap System for a Human or Animal Body

(75) Inventor: Jeanette Adriana Johanna Groot, Helmond (NL)

Correspondence Address: Mollborn Patents, Inc., 2840 Colby Drive, Boulder, CO, 80305, US

(73) Assignee: SMALL FACTORY B.V., Hellmond (NL)

(21) Appl. No.: 12/426,840

(22) Filed: Apr. 20, 2009

(30) Foreign Application Priority Data
Sep. 5, 2008 (NL) 2001957

(51) Int. Cl. A61F 5/37; A01K 29/00

(52) U.S. Cl. 128/870; 119/712

(57) ABSTRACT

A fixation wrap system for restricting a position and orientation of a human or animal body is provided. The system comprises: i) a base wrap configured for being wrapped around a desired support member, ii) a fixation wrap, and iii) releasable attachment means provided on at least one of the base wrap and the fixation wrap and being configured for releasably attaching said fixation wrap to said base wrap, and wherein said fixation wrap is configured for being wrapped around a chest and/or abdomen of said human or animal body to restrict said position and orientation with respect to said base wrap. The fact that the fixation wrap is releasably attached to the base wrap opens up the possibility to provide a fixation wrap with different sizes, each size being suitable for wrapping around a human or animal body having a different size.

### FIELD OF THE INVENTION

The invention relates to a fixation wrap system for restricting a position and orientation of a human or animal body, to a base wrap configured for use in such system, and to a fixation wrap configured for use in such system. The fixation wrap system of various embodiments of the invention is applicable in a wide variety of application areas, for example sleep wraps for babies to prevent sudden infant death syndrome (SIDS), sleep wraps for handicapped people and elderly people to prevent them falling out of their bed, fixation wraps for animals, i.e. to fix their position and orientation on a surgery table at a veterinary doctors practice.

### BACKGROUND

An infant sleep wrap system for babies is disclosed in WO93/17606. This infant sleep wrap is configured for maintaining a desired position of an infant in a bed or cot. It comprises a base member having attachment means for attachment to the base member, a securing member which is sewed to the base member and sufficiently long to encircle an infant's chest and/or abdomen and is sufficiently wide to at least extend substantially between the lateral chest and the lower abdomen of the infant. The infant sleep wrap further comprises fastening means on the securing member for fastening the securing member about the infant.

The problem with the known infant sleep wrap is that it is not suitable for a wide range of infant sizes, let alone elderly people or animals. Once the base member is attached to a desired support member such as a bed it is only possible to wrap the securing member around a baby.

### SUMMARY

The various embodiments of the invention provide a fixation wrap system that is suitable for human or animal body bodies of various different sizes, including infants, elderly people, pats such as cats and dogs, etc.

The invention is defined by the independent claims. The dependent claims define advantageous embodiments.

In a first aspect, a fixation wrap system for restricting a position and orientation of a human or animal body is provided. This system comprises i) a base wrap configured for being wrapped around a desired support member, ii) a fixation wrap, and iii) releasable attachment means provided on at least one of the base wrap and the fixation wrap and being configured for releasably attaching said fixation wrap to said base wrap, and wherein said fixation wrap is configured for being wrapped around a chest and/or abdomen of said human or animal body to restrict said position and orientation with respect to said base wrap.

The effect of the features of the fixation wrap system in accordance with various embodiments of the invention is as follows. The fact that the fixation wrap is releasably attached to the base wrap opens up the possibility to provide a fixation wrap with different sizes, each size being suitable for wrapping around a human or animal body having a different size. For example, in case an elderly person has to be attached to a bed, a fixation wrap that is configured to be wrapped around a chest and/or abdomen of the elderly person, can be attached to the base wrap. In case an infant or a baby has to be mounted to the bed, a fixation wrap that is configured to be wrapped around a chest and/or abdomen of the infant or baby, can be attached to the base wrap. The opposite is also true. The fixation wrap system opens up the possibility to provide a base wrap with different dimensions, each size being suitable for wrapping around a desired support member, or bed of a different size. It is the insight of the inventors that there is a need for such system which enables restriction of a position and orientation of a human or animal body having a wide variety of sizes while being mounted to desired support members also having a wide variety of sizes.

The fixation wrap system in accordance with various embodiments of the invention may also be used for swaddling of a baby. While doing so, the arms of the baby are also wrapped in the fixation wrap together with the baby's abdomen and/or chest.

In an embodiment of the fixation wrap system in accordance with the invention the base wrap has a substantially rectangular shape. Such a shape is the most convenient for being wrapped around a desired support member, such as a mattress, a table, a bench, a seat, or a base of a bed or cot.

In an embodiment of the fixation wrap system in accordance with the invention the width of the base wrap lies in the range between 200 mm and 1000 mm, and preferably equals about 540 mm. The width of the base wrap is defined as the smallest dimension of the rectangular shape. The width should not be too large, because otherwise the ease of use is adversely affected. Also, it should not be too small, because otherwise the mechanical strength is adversely affected (i.e. in case of use of cotton a thicker piece of cloth may be required which is less comfortable for the human or animal body). The width should also not be too small for safety reasons, i.e. the baby might get stuck under the base wrap. The width of 540 mm has been found to be an optimum width.

In an embodiment of the fixation wrap system in accordance with the invention the length of the base wrap lies in the range between 1000 mm to 5200 mm, and preferably equals about 2400 mm. The length of the base wrap is defined as the largest dimension of the rectangular shape. This range of lengths complies with dimensions of most desired support member for wrapping the base wrap around it. A length of 2400 mm is suitable for being wrapped around most mattresses of a single bed.

In an embodiment of the fixation wrap system in accordance with the invention the base wrap is provided with base wrap connection means for connecting a first end of the base wrap with a second end of the base wrap when wrapped around desired support member.

In an embodiment of the fixation wrap system in accordance with the invention the base wrap connection means are selected from a group comprising: Velcro strip, push-button/snap, zipper, and a fastener, and any combination thereof.

In an embodiment of the fixation wrap system in accordance with the invention the base wrap connection means comprise a first connecting region provided on one end of a first side on of the base wrap.

In an embodiment of the fixation wrap system in accordance with the invention the first connecting region has a size of 400 mm*160 mm.

In an embodiment of the fixation wrap system in accordance with the invention the first connecting region comprises three Velcro strips of 400*20 mm each spaced 50 mm apart.

In an embodiment of the fixation wrap system in accordance with the invention base wrap connecting means further comprise a second connecting region provided on another end on a second side of the base wrap.

In an embodiment of the fixation wrap system in accordance with the invention the second connecting region has a size of 320 mm*1250 mm.

In an embodiment of the fixation wrap system in accordance with the invention the second connecting region comprises three Velcro strips of 20*1250 mm spaced 150 mm apart.

In an embodiment of the fixation wrap system in accordance with the invention the fixation wrap comprises a main part for wrapping around said chest and/or abdomen of said human or animal body.

In an embodiment of the fixation wrap system in accordance with the invention said main part has an elongated shape. Such a shape is the most convenient for being wrapped around a human or animal body.

In an embodiment of the fixation wrap system in accordance with the invention the length of the main part lies between 500 mm and 1500 mm. The length of the base wrap is defined as the longest dimension of the rectangular shape.

In an embodiment of the fixation wrap system in accordance with the invention the length of the main part is 600 mm. This length makes the fixation wrap suitable for being wrapped around small babies, typically for baby's with an age between 0 and 3 months, but these limits are not hard.

In an embodiment of the fixation wrap system in accordance with the invention the length of the main part is 810 mm. This length makes the fixation wrap suitable for being wrapped around larger babies, typically for baby's with an age between 3 and 36 months, but these limits are not hard.

In an embodiment of the fixation wrap system in accordance with the invention the main part has in at least a middle portion thereof a width between 95 mm and 245 mm, and 150 mm. This dimension corresponds with a preferred dimension of a pocket which is part of another embodiment.

In an embodiment of the fixation wrap system in accordance with the invention the main part comprises main part connection means for connecting a first end of the main part with a second end of the main part when wrapped around desired support member.

In an embodiment of the fixation wrap system in accordance with the invention the main part connection means are selected from a group comprising: Velcro strip, push-button/snap, zipper, and fastener, and any combination thereof.

In an embodiment of the fixation wrap system in accordance with the invention the main part connection means comprise a first main part connecting region provided on one end of a first side on of the main part.

In an embodiment of the fixation wrap system in accordance with the invention the first main part connecting region has a size of 100 mm*275 mm.

In an embodiment of the fixation wrap system in accordance with the invention the first main part connecting region comprises three Velcro strips of 20 mm* 275 mm spaced apart 60 mm.

In an embodiment of the fixation wrap system in accordance with the invention the main part connecting means further comprise a second main part connecting region provided on another end on a second side of the main part.

In an embodiment of the fixation wrap system in accordance with the invention the second main part connecting region has a size of 100 mm*140 mm.

In an embodiment of the fixation wrap system in accordance with the invention the second main part connecting region comprises two Velcro strips of 100 mm* 50 mm spaced 30 mm apart.

In an embodiment of the fixation wrap system in accordance with the invention the fixation wrap comprises a further part connected with an end portion to a middle part of said main part and extending in a direction substantially perpendicular to the main part for being folded between legs of said body. The advantage of providing the fixation wrap with the further part in this manner is that sudden infant death syndrome (SIDS) is better prevented. The advantage of the further part is also that when the infant or baby is sleeping on a tilted surface (this may be required for so-called reflux babies), the baby is better kept in position and this prevents the baby to get stuck under the blankets. When folded between the legs of a baby (and connected to the main part) it is prevented that the baby rolls to a position which may be harmful.

In an embodiment of the fixation wrap system in accordance with the invention the further part of fixation wrap has elongated shape. Such a shape is the most convenient for being between legs of a human or animal body.

In an embodiment of the fixation wrap system in accordance with the invention the further part of fixation wrap has a length in a range between 300 mm and 800 mm.

In an embodiment of the fixation wrap system in accordance with the invention the further part of fixation wrap has a length of 325 mm.

In an embodiment of the fixation wrap system in accordance with the invention the further part of fixation wrap has a length of 400 mm.

In an embodiment of the fixation wrap system in accordance with the invention said further part of fixation wrap has at said end portion a width between 40 mm and 100 mm, and preferably 68 mm. Such width matches upper leg to upper leg distance of most human bodies.

In an embodiment of the fixation wrap system in accordance with the invention the further part comprises further part connection means on a further end portion for connecting to said respective main part connecting means when said main part is wrapped about said human or animal body.

In an embodiment of the fixation wrap system in accordance with the invention the further part connection means are selected from a group comprising: Velcro strip, push-button/snap, zipper, and a fastener, and any combination thereof.

In an embodiment of the fixation wrap system in accordance with the invention the further part connection means comprise a first further part connecting region provided on said further end of a first side on of the further part.

In an embodiment of the fixation wrap system in accordance with the invention the first further part connecting region has a size of 100 mm*50 mm.

In an embodiment of the fixation wrap system in accordance with the invention the first further part connecting region comprises one Velcro strip of 100 mm*50 mm.

In an embodiment of the fixation wrap system in accordance with the invention the further part connecting means further comprise a second further part connecting region provided on same further end on a second, opposing, side of the further part.

In an embodiment of the fixation wrap system in accordance with the invention the second further part connecting region has a size of 70 mm*80 mm.

In an embodiment of the fixation wrap system in accordance with the invention the second further part connecting region comprises two Velcro strips of 20 mm*80 mm spaced 30 mm apart.

In an embodiment of the fixation wrap system in accordance with the invention said releasable attachment means comprise a pocket having two pocket openings on opposite edges, wherein said pocket openings is accessible from at least one side of the base wrap for releasably receiving said fixation wrap by putting the fixation wrap through the pocket. The provision of a pocket on the main wrap, through which the fixation wrap is put, is a very convenient and safe way of providing releasable attachment means. The resulting surface is flat (i.e. no buttons or other small objects of the attachment means sticking out of the surface) which is the most convenient for the human or animal body lying on it. The pocket can be of any size and have any shape, but it must be noted that the size of the pocket in terms of distance between the pocket openings should be substantially shorter than the fixation wrap, and preferably less than one fourth of it.

In a first variant of last mentioned embodiment of the fixation wrap system said pocket comprises a piece of cloth that is attached to said base wrap at at least two, opposing, ends thereof to obtain said pocket. This embodiment provides a first way of forming a pocket on the base wrap. The advantage of this embodiment is that it provides the highest mechanical strength. In a second variant said pocket is formed by two slits in the base wrap at a certain distance from each other. This embodiment provides a second way of forming a pocket on the base wrap. The advantage of this embodiment is that it requires less material and is, due to its thinner construction also more comfortable for the human or animal lying in the fixation wrap system.

In an embodiment of the fixation wrap system in accordance with the invention said pocket has a rectangular shape. This shape matches the dimensions of the fixation wrap in accordance with other embodiments of the invention.

In an embodiment of the fixation wrap system in accordance with the invention the pocket openings have a length between 100 mm and 350 mm, and preferably 155 mm.

In an embodiment of the fixation wrap system in accordance with the invention the distance between the pocket openings lies between 100 mm and 200 mm, and preferably equals 130 mm.

In an embodiment of the fixation wrap system in accordance with the invention the piece of cloth has a width between 100 mm and 200 mm, and preferably 130 mm.

In an embodiment of the fixation wrap system in accordance with the invention the piece of cloth has a length between 140 mm and 390 mm, and preferably 195 mm.

In an embodiment of the fixation wrap system in accordance with the invention the pocket is attached to base wrap with four pocket connecting regions, one at each corner thereof. The advantage of this embodiment is that the pocket has four openings, one at each side. This allows inserting the fixation wrap into the pocket in different manners, i.e. for different orientations of the animal or human body with respect to the base-wrap/desired support member.

In an embodiment of the fixation wrap system in accordance with the invention the pocket connecting regions have a size of 18 mm*18 mm and are spaced apart from respective edges of the pocket with a distance of 2 mm. This leaves a maximum width of a middle part of the main part of the fixation wrap to be 155 mm (but preferably this is slightly less) in case of a pocket with a length of 195 mm and a width of 130 mm.

In an embodiment of the fixation wrap system in accordance with the invention the piece of cloth is sewed to base wrap at the pocket connecting regions.

In an embodiment of the fixation wrap system in accordance with the invention the pocket is mounted on the base wrap in between respective positions of the first connecting region and the second connecting region, respective positions of the respective connecting regions being defined in projection onto the base wrap.

In an embodiment of the fixation wrap system in accordance with the invention the base wrap is provided with Velcro underneath the piece of cloth and the fixation wrap is provided with receiving Velcro configured to cooperate with said Velcro when the fixation wrap is put through the pocket. In this embodiment the fixation wrap is better (more strongly) fixed to base wrap, which is advantageous in case the animal or human body tries to move.

An embodiment of the fixation wrap system in accordance with the invention further comprises a further fixation wrap and a further releasable attachment means provided on at least one of the base wrap and the further fixation wrap and spaced apart from said releasable attachment means and being configured for releasably attaching said further fixation wrap to said base wrap, and wherein said further fixation wrap is configured for being wrapped around a chest and/or abdomen of a further human or animal body to restrict said position and orientation with respect to said base wrap. This is a very interesting embodiment in case the desired support member is large enough to carry more than one human or animal body. Multiple fixation means, each specifically configured for specific size of a human or animal, can be attached to the same base wrap. For this embodiment the same features and variations are possible for the further releasable attachment means as for releasable attachment means. Also, for this embodiment the same features and variations are possible for the further fixation wrap as for fixation wrap.

An embodiment of the fixation wrap system in accordance with the invention is specifically configured for the size of a human body, and in particular for the size of a baby. This embodiment constitutes a sleep wrap for humans where the fixation wrap system of the invention is particularly useful. It may prevent elderly people to fall out of their beds and it may prevent the sudden infant death syndrome when used for babies.

In a second aspect, a base wrap is provided that is configured for use in the fixation wrap system, wherein the base wrap is configured for being wrapped around a desired support member, and wherein the base wrap is configured for being releasably attached to said fixation wrap. All features of base wrap mentioned in the system apply to base wrap in accordance with the invention. Also, the advantages of the base wrap as such follows that of the base wrap when present in the fixation wrap system.

In a third aspect, a fixation wrap is provided that is configured for use in the fixation wrap system, wherein the fixation wrap is configured for being wrapped around a chest and/or abdomen of said human or animal body to restrict said position and orientation with respect to said base wrap, and wherein the fixation wrap is configured for being releasably attached to said base wrap. All features of base wrap mentioned in the system apply to base wrap in accordance with the invention. Also, the advantages of the fixation wrap as such follows that of the fixation wrap when present in the fixation wrap system.

These and other aspects of the invention are apparent from and will be elucidated with reference to the embodiments described hereinafter.

### BRIEF DESCRIPTION OF THE DRAWINGS

In the drawings:

FIG. 1 shows a fixation wrap system in accordance with an embodiment of the invention which comprises a base wrap and a first embodiment of a fixation wrap;

FIG. 2a shows a top-view of the base wrap in FIG. 1;

FIG. 2b shows a bottom-view of the base wrap in FIG. 1;

FIG. 3 shows the base wrap of FIGS. 2a and 2b without a pocket;

FIG. 4a shows a top-view of a first embodiment of the fixation wrap in FIG. 1;

FIG. 4b shows a bottom-view of the first embodiment of the fixation wrap in FIG. 1;

FIG. 5a shows a top-view of a second embodiment of the fixation wrap;

FIG. 5b shows a bottom-view of a second embodiment of the fixation wrap;

FIG. 6a shows an embodiment of the pocket which is used as attachment means in FIG. 1,

FIG. 6b shows another embodiment of the pocket that can be used as attachment means;

FIG. 6c shows a schematic view of a fixation wrap with the other embodiment of the pocket on it; and

FIG. 7 shows a zoomed top-view of the base wrap of FIG. 2a.

### DETAILED DESCRIPTION

Various embodiments of the invention provide a fixation wrap system for restricting a position and orientation of a human or animal body, such as infants/babies, elder people, handicapped people, cats, dogs, etc. Such system comprises a base wrap to be attached to a desired object, a fixation wrap to be wrapped around a human or animal body, and attachment means for releasably attaching said base wrap to the fixation wrap. A designer has a tremendous amount of design freedom when designing the base wrap, fixation wrap and attachment means in accordance with the invention, i.e. many embodiments exist which fall within the scope of the claims. In this description embodiments for use with infants will be discussed in more detail. Nevertheless, embodiments that are suitable for other (human or animal) bodies follow the same principle. In most cases it's simply choosing other dimensions than the ones specified for the embodiment. In order to facilitate the discussion of the detailed embodiments a few expressions are defined hereinafter.

Throughout this description with “a base wrap in accordance with an embodiment of the invention” is meant a base wrap that is not permanent fixed to a fixation wrap.

Throughout this description with “a fixation wrap in accordance with an embodiment of the invention” is meant a fixation wrap that is not permanently fixed to a base wrap.

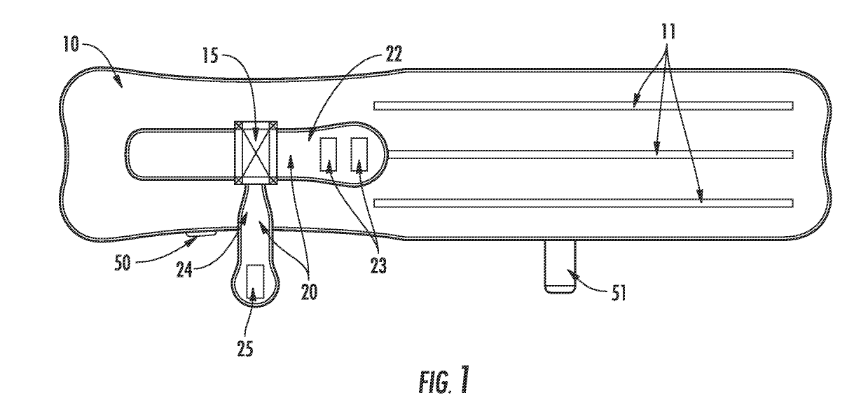

FIG. 1 shows a fixation wrap system in accordance with an embodiment of the invention. The fixation wrap system comprises a base wrap 10 and a first embodiment of a fixation wrap 20. The base wrap is made from flexible material, such as: cotton, jersey, tricot, flannel, terry cloth, linen, viscose, wool, silk, polyester, leather, suede, satin, and denim. The flexibility of the material allows the wrap to be wrapped around a desired object, such as a mattress, a table, a bench, a seat, or a base of a bed or cot. The base wrap 10 in the embodiment of FIG. 1 is provided with a first base wrap connecting region 11 on one end and a second base wrap connecting region (not shown) on the other end. The first base wrap connecting region 11 serves to connect both ends of the base wrap with each other when wrapped around the desired object. Optionally, the main wrap 10 may be provided with labels 50, 51 attached thereto for carrying a trademark or trade name. In FIG. 1 the main wrap 10 is provided with a pocket 15 for releasably receiving the fixation wrap 20. The fixation wrap comprises a main part 22 which is designed with such dimensions that it can be wrapped around a chest and/or abdomen of a baby having an age between 0 and 3 months. The fixation wrap 20 further comprises a further part 24 which is connected to a middle part of the main part 22 and serves to be folded, in operational use, between the legs of a baby and attached to the main part above the abdomen and/or chest of the baby. The fixation wrap 20 is made from flexible material, such as: cotton, jersey, tricot, flannel, terry cloth, linen, viscose, wool, silk, polyester, leather, suede, satin, and denim. The main part 22 of the fixation wrap 20 in the embodiment of FIG. 1 is provided with a first main part connecting region 23 on one end and a second main part connecting region (not shown) on the other end. These connecting regions serve to connect both ends of the main part 22 of the fixation wrap 20 with each other when wrapped around the baby's chest and/or abdomen. The further part 24 of the fixation wrap 20 is also provided with a first further part connecting region 25 which serves for attaching the further part 24 to the main part 22 above the abdomen and/or chest of the baby in operational use. The fixation wrap system of FIG. 1 is specifically designed for use with infants/babies and serves to ensure their comfort and safety, and to prevent SIDS. An important advantage of the fixation wrap system of FIG. 1 is that the separate parts can be detached from each other which makes them easy to wash and iron.

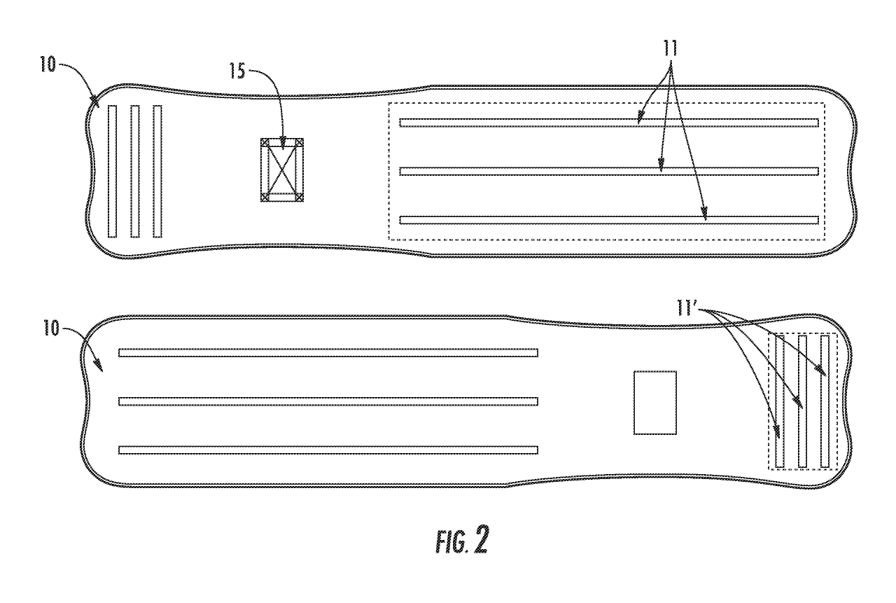

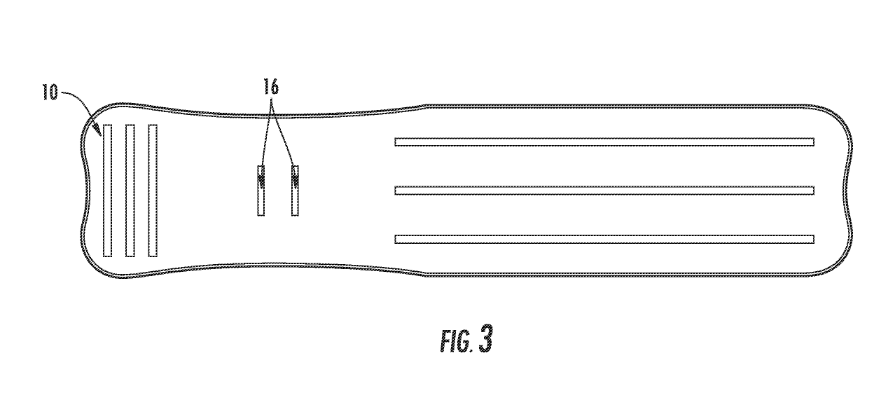

FIG. 2a shows a top-view of the base wrap 10 in FIG. 1. FIG. 2b shows a bottom-view of the base wrap 10 in FIG. 1. The base wrap 10 has an elongated shape with a base wrap length Lb of 2400 mm and a base wrap width Wb of 540 mm. In another embodiment, the base wrap width is 700 mm. The base wrap 10 is provided with a pocket 15 having a pocket width Wp of 130 mm and a pocket length Lp of 195 mm. The pocket 15 is placed at a first distance Dp1 from the left side (in top-view) which equals 550 mm. Further, the pocket 15 is placed at a second distance Dp2 from the upper-side and lower-side (in top-view) which equals 172.5 mm. The first base wrap connecting region 11, which is provided on the top-side of the base wrap 10, comprises three strips of Velcro, each strip having a length of 1250 mm and a width of 20 mm. These strips 11 extend in the same direction as the base wrap 10 and are equally spaced apart over a distance of 150 mm The second base wrap connecting region 11′, which is provided on the bottom-side of the base wrap 10, comprises three strips of receiving Velcro, each strip having a length of 400 mm and a width of 20 mm. These strips 11′ extend in a direction perpendicular to the direction in which the base wrap 10 extends and are equally spaced apart over a distance of 50 mm. The strips of receiving Velcro 11′ on the bottom side can be attached to the strips of Velcro 11 on the top side when the base wrap 10 is wrapped around a support member, such as a mattress.

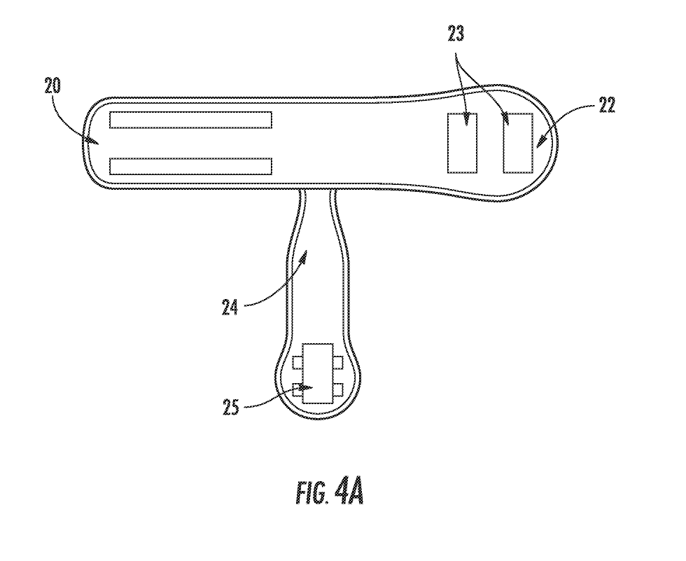

FIG. 3 shows the base wrap 10 of FIGS. 2a and 2b without a pocket. In this figure a pair of Velcro strips 16 is shown which are located at the location where the pocket is to be provided. The Velcro strips 16 each have a length of 150 mm and a width of 20 mm, such that they fall completely within the pocket 15, which is provided later. The fixation wrap (not shown in this figure) which is to be put through into the pocket may be provided with strips of receiving Velcro (on its bottom-side) such that the fixation wrap is more firmly attached to the base wrap 10 in operational use. An advantage is that, in case of use for a baby, the baby's position is better fixed, which may prevent the baby to roll to another position which is harmful for the baby.

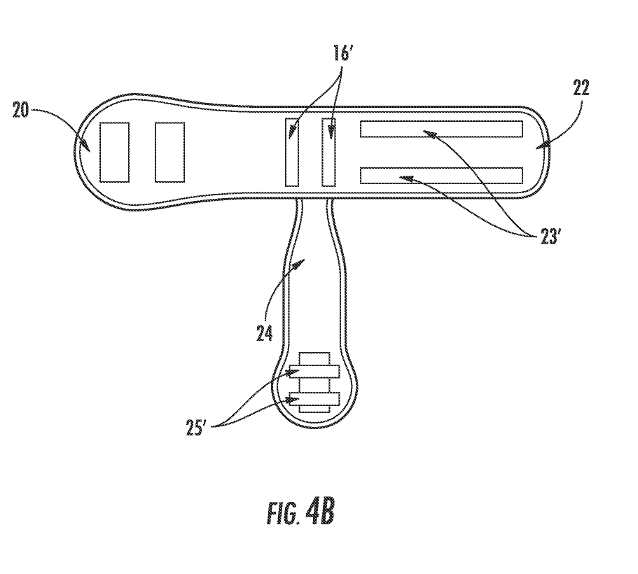

FIG. 4a shows a top-view of a first embodiment of the fixation wrap 20 in FIG. 1. FIG. 4b shows a bottom-view of the first embodiment of the fixation wrap 20 in FIG. 1. This embodiment is particularly suitable for infants with an age between 3 months and 36 months. The fixation wrap 20 comprises the main part 22 and the further part 24 connected to a middle portion of the main part 22. The main part 22 has a substantially elongated shape with a length Lmp of 810 mm. At an end (on the left side in the figure) the main part 22 has a first width Wmp1 of 150 mm. The width of the main part 22 gradually increases towards a broadened part at the other end (at the right side of the figure) and at the other end the main part 22, wherein the broadened part has a maximum width Wmp2 of 200 mm. The further part 24 has a substantially elongated shape with a length Lfp of 400 mm. At an end that is in contact with the middle portion of the main part (in the center of the figure) the further part 24 has a first width Wfp1 of 68 mm. The width of the further part 24 gradually increases towards a broadened part at the other end (at the bottom side of the figure). In the middle portion of the further part the width is about 86 mm, and the broadened part at the other end has a maximum width Wfp3 of 146 mm.

The first main part connecting region 23, which is provided on the top-side of the main part 22 of the fixation wrap 20, comprises two strips of Velcro, each strip having a length of 100 mm and a width of 50 mm. These strips extend in a direction perpendicular to the direction in which the main part 22 extends and are equally spaced apart over a distance of 30 mm. The second main part connecting region 23′, which is provided on the bottom-side of the main part 22 of the fixation wrap 20, comprises two strips of receiving Velcro, each strip having a length of 275 mm and a width of 20 mm. These strips extend in the same direction as the main part 22 and are equally spaced apart over a distance of 70 mm. The strips of receiving Velcro 23′ on the bottom side can be attached to the strips of Velcro 23 on the top side when the main part 22 is wrapped around the baby's chest and/or abdomen.

The first further part connecting region 25, which is provided on the top-side of the further part 24 of the fixation wrap 20, comprises one strip of Velcro having a length of 100 mm and a width of 50 mm. This strip extends in the same direction as the further part 23. A second further part connecting region 25′ is provided on the bottom-side of the further part 24 of the fixation wrap 20, and comprises two strips of receiving Velcro, each strip having a length of 80 mm and a width of 20 mm. These strips extend in a direction perpendicular to the direction in which the further part 24 extends and are equally spaced apart over a distance of 35 mm. The strip of Velcro 25 on the top side of the further part 24 can be attached to the strips of receiving Velcro 23′ on the bottom side of the main part 22, when the further part 24 is folded between legs of the baby's to be connected to the main part, which has been wrapped around the chest and/or abdomen of the baby. Likewise, the strips of receiving Velcro 25′ on the bottom side of the further part 24 can be attached to the strips of Velcro 23 on the top side of the main part 22, when the further part 24 is folded between legs of the baby's to be connected to the main part 22 that has been wrapped around the chest and/or abdomen of the baby. It must be noted that the end of the main part 22 with the broadened part goes on top of the other end while the further part is sandwiched between them.

On a middle portion of the bottom side of the main part 22 (between locations of said first and second main part connecting regions 23, 23′) two strips of receiving Velcro (16′) are provided for being attached to the earlier mentioned pair of Velcro strips 16 to establish a more firm connection between the base wrap 10 and the fixation wrap 20.

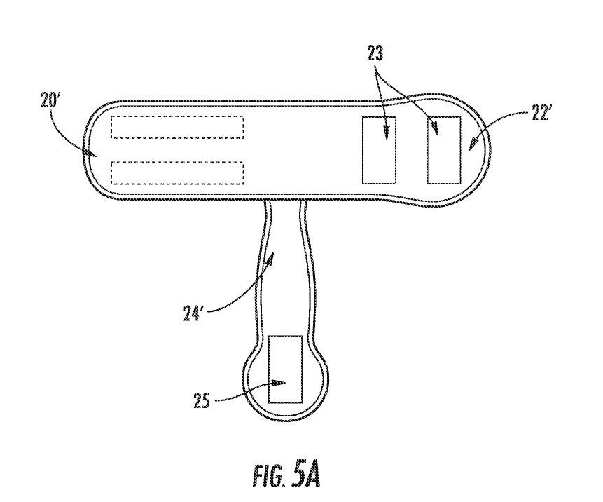

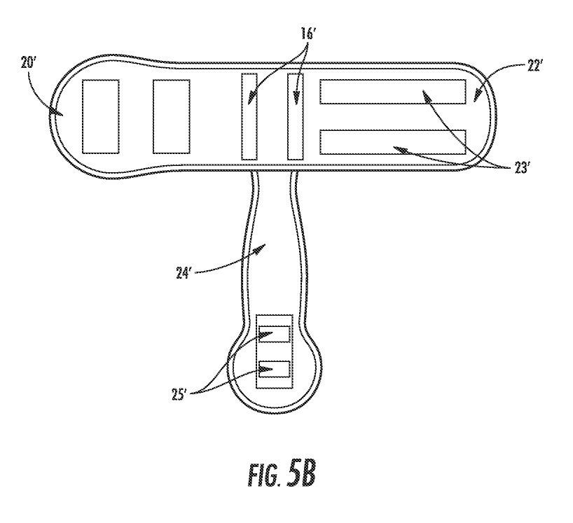

FIG. 5a shows a top-view of a second embodiment of the fixation wrap 20′. FIG. 5b shows a bottom-view of a second embodiment of the fixation wrap 20′. This embodiment is smaller than the embodiment of FIGS. 4a and 4b, and particularly suitable for infants with an age between 0 months and 3 months. Most elements of the second embodiment are the same as for the first embodiment of FIGS. 4a and 4b. Therefore, the second embodiment will be discussed in as far as it differs from the first embodiment only. A first difference is that the main part 22′ has a length Lmp′ that is a bit shorter, namely 600 mm instead of 810 mm. Second, a second width Wmp2′ of the main part 22′ is a bit smaller, namely 175 mm instead of 200 mm. Third, a length Lmp2′ of the further part 24′ is a bit smaller, namely 325 mm instead of 400 mm. Fourth, the strips of receiving Velcro 23′ on the bottom side of the main part 20′ are a bit shorter, namely 175 mm, and they are spaced apart by a distance of 60 mm.

The fixation wraps of FIGS. 5a and 5b are made from flexible material, such as: cotton, jersey, tricot, flannel, terry cloth, linen, viscose, wool, silk, polyester, leather, suede, satin, and denim. The main part 22 and the further part 24 may be separate pieces attached together or they may be made out of one piece.

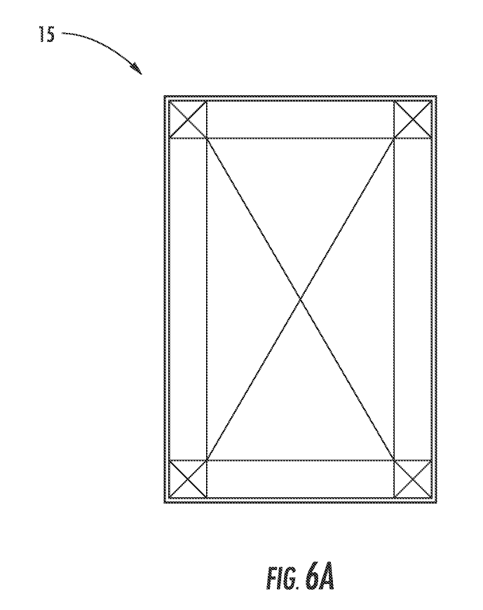

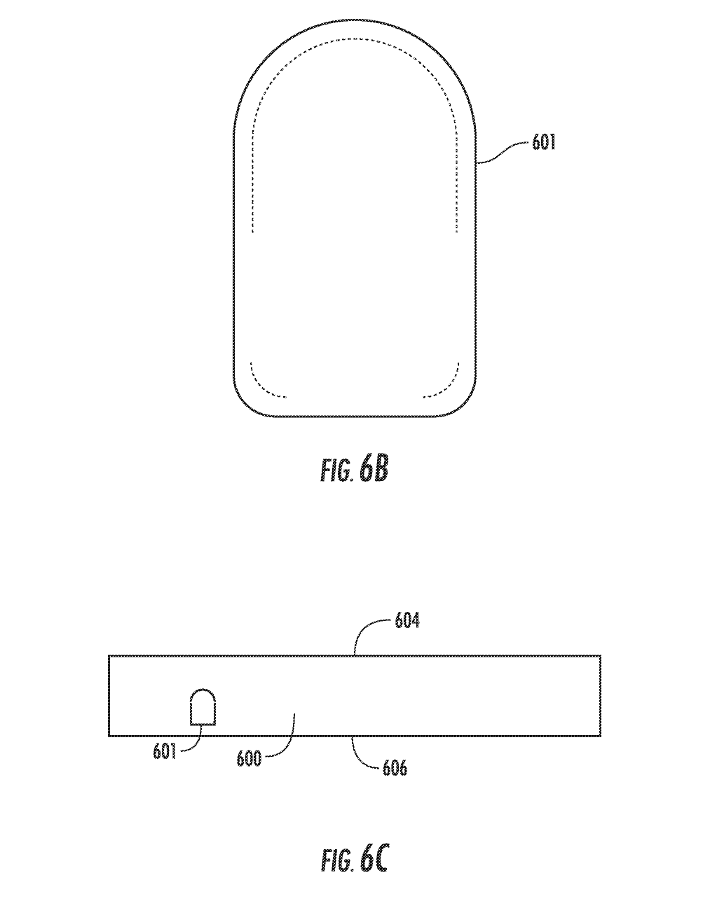

FIG. 6a shows an embodiment of the pocket 15 which is used as attachment means in FIG. 1. The pocket 15 has a rectangular shape with a length Lp of 195 mm and a width Wp of 130 mm. On each corner is provided a pocket connecting region Pcr at which the pocket 15 is attached to the base wrap 10, i.e. by sewing. All pocket connecting regions Pcr have a width Wpcr of 18 mm and a length Lpcr of 18 mm. The pocket connecting region Pcr are spaced from edges (two short and two long edges) of the pocket 18 with a distance Ds of 2 mm. In this way, a pocket 15 is obtained having four openings, i.e. two openings on the long edges having a width Wo1 of 155 mm and two openings on the short edges having a width Wo2 of 90 mm.

FIG. 6b shows another pocket 601 as another embodiment of releasable attachment means on a fixation wrap. The other pocket 601 has a round top and round bottom corners that are preferably sewed to the fixation wrap at locations indicated at a first pocket connection region Pcr1, a second pocket connection region Pcr2 and a third pocket connection region Pcr3. An advantage of this embodiment is that by using round corners, stress along the sewing stitches is better distributed, resulting in a more robust structure.

FIG. 6c shows the other pocket 601 on another base wrap 600. Whereas in previous embodiment a pocket has been shown being centered on a base wrap, the other pocket 601 is attached to the other base wrap with a first distance between the lower part of the pocket 601 and a lower side 606 of the base wrap 600 being smaller than a second distance between the upper part of the pocket 601 and a higher side 604 of the base wrap 600. It will be apparent to a person skilled in the art that the pocket 601 can also be placed in the middle of the base wrap 600. In addition, also the horizontal position of the pocket 601 on the base wrap 600 can be varied; it can also be located in or around the middle of the base wrap 600.

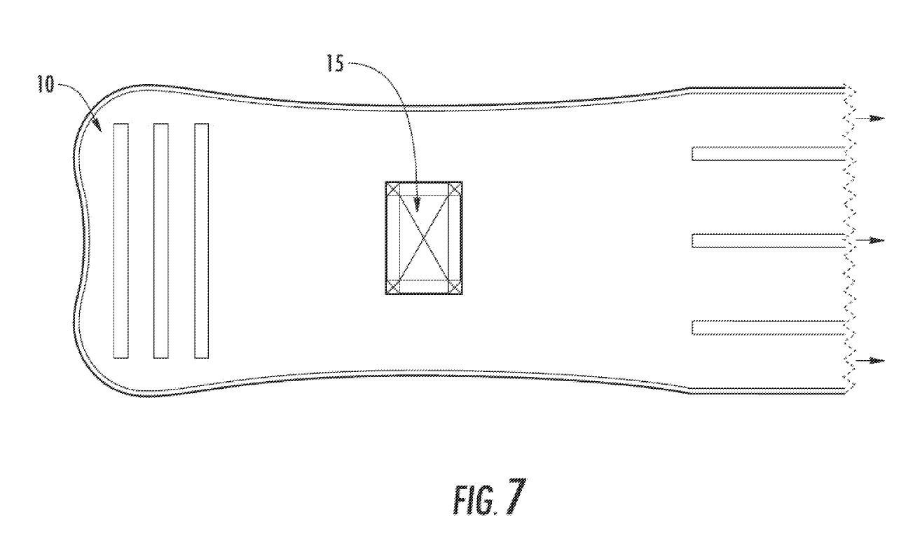

FIG. 7 shows a zoomed top-view of the base wrap 10 of FIG. 2a. This figure serves to illustrate different bed or cot sizes for which the base wrap 10 in accordance with the invention is suitable. A smallest size is that of a cradle having a width Wc of 400 mm. A second, larger, size is that of a small cot having a width Wi1 of 600 mm. A third, larger, size is that of a larger cot having a width Wi2 of 700 mm. A fourth, larger, size is that of a bed

having a width Wa1 of 800 mm. A fifth, largest size, is that of a bed having a width Wa2 of 900 mm.

Various embodiments of the invention thus provide a fixation wrap system for restricting a position and orientation of a human or animal body. The system comprises i) a base wrap (10) configured for being wrapped around a desired support member, ii) a fixation wrap (20), and iii) releasable attachment means (15) provided on at least one of the base wrap (10) and the fixation wrap (20) and being configured for releasably attaching said fixation wrap (20) to said base wrap (10), and wherein said fixation wrap (20) is configured for being wrapped around a chest and/or abdomen of said human or animal body to restrict said position and orientation with respect to said base wrap (10). Various embodiments of the invention further provide a fixation wrap configured for use such system and to a base wrap configured for use in such system. The fact that the fixation wrap is releasably attached to the base wrap opens up the possibility to provide a fixation wrap with different sizes, each size being suitable for wrapping around a human or animal body having a different size. Also, the fixation wrap system opens up the possibility to provide a base wrap with different dimensions, each size being suitable for wrapping around a desired support member, or bed of a different size.

The various embodiments of the invention may be applied in a wide variety of application areas, for example in hospitals, old-people's home's, veterinary practices, maternity hospitals and many other areas where it is desired to restrict a position and/or orientation of human and/or animal body.

Various variations of the fixation wrap system, base wrap, and fixation wrap in accordance with embodiments of the invention are possible and do not depart from the scope of the invention as claimed. These variations for example relate to dimensions, materials, shapes, etc.

In particular, even though embodiments of the invention have been described in high detail with respect to various measures of the base wrap and fixation wrap, a person skilled in the art will readily appreciate that those measures have only been provided for illustrative reasons and that measures can be varied without departing from the scope of the invention. The measures can be adapted to the specific use demands of a fixation wrap system according to the invention, based for example on experience or calculations, to accommodate certain demands with respect to the bill of materials or to the object for which the fixation wrap system will be used. A person skilled in the art will readily appreciate that the dimensions of beds an mattresses as well as of other supports vary a lot in the field and that also people and animals vary in size.

An important variation concerns the provision of the attachment means. In the examples given the attachment means, e.g. a pocket, are provided on the base wrap. However, the attachment means may also be provided on the fixation wrap or on both the base wrap and the fixation wrap. Providing attachment means such as a pocket on the fixation wrap, however, is not very practical as the base wrap is for many embodiments much larger than the fixation wrap.

Another variation concerns the materials used for the base wrap and the fixation wrap. The skilled person may easily come up with other materials which are suitable.

Another variation concerns the fixation wrap. The examples in the figure description all concerned a fixation wrap for a baby or infant. However, fixation wraps may also be made for elderly people, handicapped people, and even animals (i.e. cats and dogs).

Another variation concerns the base wrap. The base wrap discussed in the description is suitable to be wrapped around a bed ranging from a cot to a 90 cm one-person bed. However, the base wrap may also be made larger such that it can be wrapped around a 2-person bed.

Another embodiment of the invention concerns a fixation wrap system having a base wrap and multiple fixation wraps releasably attached to the base wrap. This embodiment is advantageous, because multiple human or animal bodies may be attached to a desired support member, such as a bed or a couch.

Other variations concern the attachment of a sleeping bag or swaddling band to the base wrap of the invention. When a baby is wrapped in the fixation wrap it is also possible to mount a spit cloth to the base wrap. The releasable attachment means may be re-used for that purpose.

In yet another embodiment of the invention, the bands of Velcro can be turned over and angle of 90 degrees, followed by adjusting the length and/or width of the Velcro to ensure practical use of the fixation wrap system.

In all, it will be apparent to a person skilled in the art that the invention may be embodied in numerous ways by combining various embodiments of the invention as disclosed in the description or other measures known by a person skilled in the art, even in ways that have not been disclosed above.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. In the device claim enumerating several means, several of these means may be embodied by one and the same item of hardware. The mere fact that certain measures are recited in mutually different dependent claims does not indicate that a combination of these measures cannot be used to advantage. Throughout the Figures, similar or corresponding features are indicated by same reference numerals or labels.

1. A fixation wrap system for restricting a position and orientation of a human or animal body, the system comprising: i) a base wrap configured for being wrapped around a desired support member, ii) a fixation wrap, and iii) releasable attachment means provided on at least one of the base wrap and the fixation wrap and being configured for releasably attaching said fixation wrap to said base wrap, and wherein said fixation wrap is configured for being wrapped around a chest and/or abdomen of said human or animal body to restrict said position and orientation with respect to said base wrap.

2. The fixation wrap system of claim 1, wherein the fixation wrap comprises a main part for wrapping around said chest and/or abdomen of said human or animal body.

3. The fixation wrap of claim 2, wherein said main part has an elongated shape.

4. The fixation wrap system of claim 2, wherein the fixation wrap comprises a further part connected with an end portion to a middle part of said main part and extending in a direction substantially perpendicular to the main part for being folded between legs of said body.

5. The fixation wrap system of claim 4, wherein said further part of fixation wrap has at said end portion a width between 40 mm and 100 mm, and preferably 68 mm.

6. The fixation wrap system of claim 1, wherein said releasable attachment means comprise a pocket having two pocket openings on opposite edges, wherein said pocket openings is accessible from at least one side of the base wrap for releasably receiving said fixation wrap by putting the fixation wrap through the pocket.

7. The fixation wrap system of claim 6, wherein said pocket comprises a piece of cloth that is attached to said base wrap at at least two, opposing, ends thereof to obtain said pocket.

8. The fixation wrap system of claim 6, wherein said pocket is formed by two slits in the base wrap at a certain distance from each other.

9. The fixation wrap system of claim 7, wherein said pocket has a rectangular shape.

10. The fixation wrap system of claim 6, wherein the base wrap is provided with Velcro underneath the piece of cloth.

11. The fixation wrap system of claim 10, wherein the fixation wrap is provided with receiving Velcro configured to cooperate with said Velcro when the fixation wrap is put through the pocket.

12. The fixation wrap system of claim 1, further comprising a further fixation wrap and a further releasable attachment means provided on at least one of the base wrap and the further fixation wrap and spaced apart from said releasable attachment means and being configured for releasably attaching said further fixation wrap to said base wrap, and wherein said further fixation wrap is configured for being wrapped around a chest and/or abdomen of a further human or animal body to restrict said position and orientation with respect to said base wrap.

13. The fixation wrap system of claim 1, wherein the fixation wrap system is specifically configured for the size of a human body, and in particular for the size of a baby.

14. A base wrap configured for use in the fixation wrap system of claim 1, wherein the base wrap is configured for being wrapped around a desired support member, and wherein the base wrap is configured for being releasably attached to said fixation wrap.

15. A fixation wrap configured for use in the fixation wrap system of claim 1, wherein the fixation wrap is configured for being wrapped around a chest and/or abdomen of said human or animal body to restrict said position and orientation with respect to said base wrap, and wherein the fixation wrap is configured for being releasably attached to said base wrap.
